(19) United States
(12) Patent Application Publication
(10) Pub. No.: US 2021/0322652 A1
(43) Pub. Date: Oct. 21, 2021

(54) INJECTABLE HYDROGELS FOR LOCAL DELIVERY TO THE HEART

(71) Applicant: THE TRUSTEES OF THE UNIVERSITY OF PENNSYLVANIA, Philadelphia, PA (US)

(72) Inventors: Brendan Patrick PURCELL, Brooklyn, NY (US); Jason Alan BURDICK, Philadelphia, PA (US); Leo L. WANG, Philadelphia, PA (US)

(21) Appl. No.: 17/271,778

(22) PCT Filed: Aug. 30, 2019
(86) PCT No.: PCT/US2019/049039

§ 371 Date: Feb. 26, 2021

Related U.S. Application Data
(60) Provisional application No. 62/725,404, filed on Aug. 31, 2018.

(51) Int. Cl. A61L 31/16; A61L 31/18; A61L 31/14; A61L 31/04

(52) U.S. Cl. CPC A61L 31/16; A61L 31/18; A61L 31/145; A61L 2300/232; A61L 2300/424; A61L 2300/80; A61L 31/042

(57) ABSTRACT

The invention concerns methods of delivering a hydrogel to the heart, comprising: introducing a hydrogel composition into a subject, said hydrogel comprising components mixed prior to introduction; the introducing being performed such that the hydrogel composition resides between the epicardium and the pericardium of the subject. In some embodiments, the injection is performed using a syringe or catheter.

### CROSS REFERENCE TO RELATED APPLICATIONS

This application claims benefit of U.S. Patent Application No. 62/725,404, filed Aug. 31, 2018, the disclosure of which is incorporated herein by reference in its entirety.

### GOVERNMENT RIGHTS

The invention was made with government support under Grant No. R01HL137365 and 1R41HL140645-01A1 awarded by the NIH. The government has certain rights in the invention.

### TECHNICAL FIELD

The invention concerns injectable hydrogels for delivering one or more therapeutics to the heart.

### BACKGROUND

Intramyocardial injections of hydrogels have been widely explored to locally deliver therapeutic molecules to diseased heart tissue or to themselves induce a biological or mechanical response [Tous, E., et al., Injectable acellular hydrogels for cardiac repair. J Cardiovasc Transl Res. October;4(5):528-42 2011]. These materials are in situ crosslinking or shear thinning polymer networks that can be delivered through a needle and into the myocardium. Once in the myocardium, these gels may act as depots to slowly elute active therapeutic agents to the adjacent myocardium. Numerous preclinical studies demonstrate that these materials can effectively localize active concentrations of therapeutic molecules to diseased heart tissue, leading to improved functional outcomes [Ryu, J. H., et al., Implantation of bone marrow mononuclear cells using injectable fibrin matrix enhances neovascularization in infarcted myocardium. 26:3: 319-326, 2005; Segers, V. F. M., et al., Local Delivery of Protease-Resistant Stromal Cell Derived Factor-1 for Stem Cell Recruitment After Myocardial Infarction. Circulation 116:1683-1692 2007; Purcell, B. P., et al., Injectable and bioresponsive hydrogels for on-demand matrix metalloproteinase inhibition. Nat Mater. June;13(6):653-61 2014; Wang, L. L., et al., Sustained miRNA Delivery from an Injectable Hydrogel Promotes Cardiomyocyte Proliferation and Functional Regeneration after Ischaemic Injury, Nature Biomedical Engineering, 1: 983-992, 2017; Chen, C. W., et al., Sustained Release of Endothelial Progenitor Cell-Derived Extracellular Vesicles from Shear-Thinning Hydrogels Improves Angiogenesis and Promotes Function after Myocardial Infarction, Cardiovascular Research, 114:1029-1040, 2018]. However, several obstacles remain towards clinical translation of these materials including (1) the development of catheter technologies to introduce hydrogels in a minimally invasive manner, and (2) safely injecting the materials into the myocardium without compromising the myocardial wall. Therefore, while the application of hydrogels to locally deliver therapeutic agents to diseased heart tissue is promising, safely localizing these materials in the heart remains challenging.

As an alternative to intramyocardial hydrogel injections, hydrogels have been applied to the outer surface of the heart (epicardium). For example hydrogel constructs such as fibrin have been placed or sutured onto the epicardium in pre-clinical animal studies [Liu, J., et al., Autologous stem cell transplantation for myocardial repair. Am J Physiol Heart Circ Physiol 287: H501-H511, 2004; Zhang, G. et al., Controlled release of stromal cell-derived factor-1 alpha in situ increases c-kit+cell homing to the infarcted heart, Tissue Eng. August;13(8):2063-71, 2007]. Further, an implantable pouch designed to contain hydrogels with encapsulated therapeutics has been sutured onto the epicardium in pre-clinical animal studies [Whyte, W., et al., Sustained release of targeted cardiac therapy with a replenishable implanted epicardial reservoir, Nature Biomedical Engineering, 2:416-428, 2018]. While widely explored to demonstrate proof-of-concept of locally delivered therapeutics to treat heart disease, this approach of placing or suturing materials onto the epicardium is limited to invasive procedures such as a thoracotomy to successfully implant and secure the materials to the heart.

Similarly, hydrogel sprays or precursor solutions have been developed to apply to the epicardium for local delivery of therapeutic agents. The sprays contain crosslinking polymers that are mixed shortly before spraying and solidify on the surface of the heart to form an adherent hydrogel [Feng, X. D., et al., Effectiveness of biatrial epicardial application of amiodarone-releasing adhesive hydrogel to prevent postoperative atrial fibrillation, The Journal of Thoracic and Cardiovascular Surgery, Vol. 148, No. 3, 2014]. Hydrogels are adhered through the combination of physical interdigitation with the tissue during crosslinking as well as bond formation between the hydrogel polymers and the tissue [Liu, J., et al., Autologous stem cell transplantation for myocardial repair. Am J Physiol Heart Circ Physiol 287: H501-H511, 2004; Purcell, B. P., et al., Synergistic effects of SDF-1α chemokine and hyaluronic acid release from degradable hydrogels on directing bone marrow derived cell homing to the myocardium. Biomaterials 33(31):7849-7857, 2012]. While this approach has successfully localized hydrogels to targeted regions of the heart, invasive surgical procedures are required to apply the hydrogels and require the removal of the pericardium—an elastic sac that surrounds the heart.

The pericardium is a double walled, fibrous collagen membrane that is wrapped around the heart. The pericardium and outer surface of the myocardium (epicardium) are separated by a pericardial cavity filled with fluid that provides lubrication for the beating heart within the sac and protects the heart from chest infections and physical shock. Pericardial injections, aspirations and access to targeted regions of the myocardium with tissue ablation devices and electrical mapping devices are routinely performed in the clinic through a minimally invasive subxiphoid access procedure.

Recently, a catheter device was developed that forms a circular boundary region in the pericardial cavity in which complimentary polymers are dispensed, which then mix and crosslink into a hydrogel [Garcia, J. R., et al., A Minimally Invasive, Translational Method to Deliver Hydrogels to the Heart Through the Pericardial Space, JACC: Basic to Translational Science, Vol. 2, No. 5, 2017]. Creating the boundary is critical as the uncrosslinked polymers would freely disperse within the pericardial cavity upon injection. Further, mixing and crosslinking the polymers too early within the lumen of the device would cause the device to clog, which would be detrimental during a clinical procedure.

There is a need in the art for an improved delivery system.

### SUMMARY

In some aspects, the invention concerns methods of delivering a hydrogel to the heart, comprising: introducing a hydrogel composition into a subject, said hydrogel comprising components mixed prior to introduction; the introducing being performed such that the hydrogel composition resides between the epicardium and the pericardium of the subject. In some embodiments, the injection is performed using a syringe or catheter.

Some hydrogels have a storage modulus (G′) greater than about 10 Pa. Certain hydrogels have a storage modulus (G′) is greater than about 100 Pa or 400 Pa. In some embodiments, at least a portion of the hydrogel crosslinking is performed prior to injecting. Some preferred hydrogel compositions are shear-thinning.

In some embodiments, the hydrogel is injected with an injection force is less than 50N. In other embodiments, the injection force is less than 25N.

Certain methods comprise contacting two or more components to form the hydrogel composition. In some embodiments, the contacting is performed in a mixer such that one hydrogel component is fed to the mixer through a first lumen of a catheter or syringe and a second hydrogel component is fed to the mixer through a second lumen of a catheter or syringe. In some preferred embodiments, a medicament is fed to the mixer by either the first or second lumen of a catheter or syringe. Certain methods use a third lumen of a catheter or syringe to feed the medicament to the mixer.

In some preferred compositions, the hydrogel comprises at least one of modified or unmodified gelatin, hyaluronic acid, dextran, polyvinylpyrrolidone; methlycellulose and methlycellulose derivatives; polysaccharides; alginate; chitosan or polyethylene glycol and crosslinking agent.

Certain preferred hydrogel compositions comprise a medicament of one or more of small molecule pharmaceuticals, peptides, cytokines, proteins, polysaccharides, synthetic polymers, particles, DNA plasmids, mRNA, cells, cellular exosomes, lipid nanoparticles and microparticles. Some medicaments comprise one or more of matrix metalloproteinase (MMP) inhibitors; hydroxymates; tetracyclines, minocycline; peptide based inhibitors; ion chelators. In some embodiments, the MMP inhibitor comprises one or more of recombinant tissue inhibitor of MMPs (TIMPs) selected from TIMP-1, TIMP-2, TIMP-3 and TIMP-4. In certain embodiments, the hydroxymate comprises illomastat. Some tetracyclines comprise one or more of doxycycline, modified doxycyclines, and minocycline.

In some embodiments, the medicament comprises one or more of (i) miRNA; (ii) siRNA; (iii) plasmid DNA; (iv) growth factors; (v) heat shock proteins; (vi) cytokines; (vii) cells; and (viii) cellular vesicles/exosomes. In some embodiments, the medicament comprises one or more of semi-synthetic sulfated polysaccharides such as pentosan polysulfate, sulfated hyaluronic acid and dextran sulfate. Certain embodiments have a medicament comprising one or more of naturally sulfated polysaccharides such as heparin and chondroitin sulfate. Some compositions have a medicament comprising one or more of (i) steroids and (ii) anti-inflammatory compounds. In yet other embodiments, the medicament comprises one or more metal ion chelators.

In some aspects, the methods are used to treat myocardial infarction, heart failure, atrial fibrillation, coronary artery disease, aetherosclerosis, angina, aneurysms, hypertension, rheumatic heart disease, cardiac arrest, ischemia, congestive heart failure, arrhythmia, congenital heart diseases, cardiomegaly, heart valve diseases, cardiomyopathy, dilated cardiomyopathy, hypertrophic cardiomyopathy, restrictive cardiomyopathy, pericarditis, pericardial effusion, marfan syndrome, heart murmers, or post surgical tissue repair.

In yet another aspect, the hydrogel composition prevents the formation of post-operative adhesions.

Certain embodiments utilize a hydrogel composition comprising a radiopaque material to guide the introduction of the hydrogel. Suitable radiopaque materials include iohexol or zirconia.

Some hydrogels contain chemical modifications to enhance adhesion to the epicardium or pericardium. In certain embodiments, the adhesive groups are aldehyde, catechol, or gallol groups.

In yet another aspect, the hydrogel stimulates the formation of tissue including fibrous tissues to mechanically support the myocardial wall or functional cardiac tissue to contribute to cardiac function.

### BRIEF DESCRIPTION OF THE DRAWINGS

FIG. 1 illustrates that injectable hydrogels can be passed through a syringe with clinically feasible injection forces and form a pocket in the pericardial cavity due to pericardial tensile forces and hydrogel crosslink forces.

FIG. 2 illustrates shear-thinning dextran-aldehyde/gelatin-hydrazide gels (704 Pa) containing blue food coloring injected into the pericardial cavity to form a pocket with a defined boundary. Saline containing blue food coloring quickly dispersed throughout the pericardial cavity in an uncontrollable fashion.

FIG. 3 illustrates shear thinning HA-aldehyde/HA-hydrazide hydrogels containing blue food coloring being injected into the pericardial cavity of an ex-vivo beating heart model.

FIG. 4 illustrates shear thinning HA-aldehyde/HA-hydrazide hydrogels being explanted from the pericardial cavity after 3 hrs of being exposed to myocardial wall motion.

FIG. 5 illustrates shear thinning HA-aldehyde/gelatin-hydrazide hydrogels containing blue food coloring being injected into the pericardial cavity of explanted hearts and subjected to a shear motion of the pericardium.

FIG. 6 shows the adhesion of HA-aldehyde/gelatin-hydrazide gels to the epicardium as measured by gluing strips of epicardial tissue together and measuring the force to failure with a tensile test.

FIG. 7 shows HA-aldehyde/gelatin-hydrazide gels being injected into the pericardial cavity at different times during the gelation process.

FIG. 8 shows an example where the syringe and catheter were loaded onto an Instron, and the injection force was measured with compressive ramps of 15 mm/min and 30 mm/min until the syringe was empty.

FIG. 9 illustrates an example where the hydrogel was loaded in a catheter and was inserted into the pericardium and 0.3 ml of the hydrogel was injected at the right ventricle apex (Site 1), lateral/FW right atrium (Site 2), left atrium appendage region (Site 3) and lateral left ventricle (Site 4).

FIG. 10 shows an example where the hydrogel gradually spreads to form a single patch around the injection site in vivo.

FIG. 11 shows an example of a targeted percutaneous injection of a hydrogel patch within the pericardial cavity using fluoroscopy. Hydrogel boundary is identified with white arrows (scale bar=3 cm)

FIG. 12 shows an example of an adherent hydrogel patch three days after percutaneous injection (scale bar=2 cm).

FIG. 13 shows an example of the local myocardial delivery of a fluorescent molecule encapsulated in the hydrogel patch (scale bars=1.5 cm).

### DETAILED DESCRIPTION OF ILLUSTRATIVE EMBODIMENTS

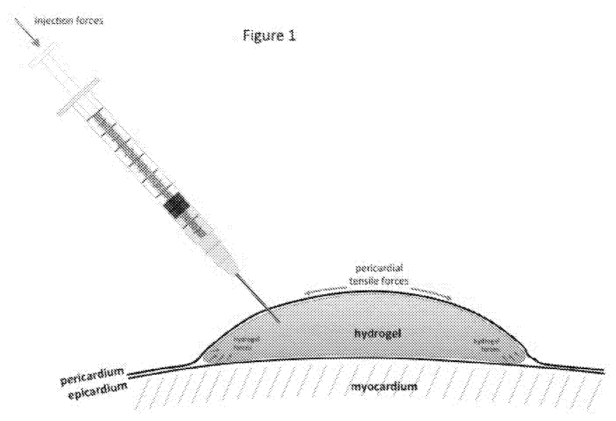

It is challenging to introduce hydrogels to the heart's pericardial cavity in an effective and safe matter. Here, we report the discovery that injectable hydrogels with sufficient strength can be injected through a syringe and maintain a clear boundary within the pericardial cavity through a simple procedure using commercially available catheters. Upon injection, hydrogels form a pocket between the epicardium and elastic pericardium (FIG. 1). Surprisingly, we have found that gels with storage moduli (G′) as low as 10 Pa have sufficient hydrogel forces to create a clearly defined boundary within the pericardial cavity. It is anticipated that hydrogels with lower storage moduli may also create a clear boundary at the injection site for localization of the hydrogel, but solutions with physical properties that approach the viscosity of saline will quickly disperse from the injection site. The hydrogel forces that contain the polymers within the injection site boundary are due to polymer entanglements and crosslinks, both physical or covalent. While these relatively weak gels create a boundary upon injection, gel moduli greater than 5, 8 or 10 Pa are preferred to withstand shear forces and compression forces within the pericardial cavity during heart beating and movement of the pericardium. More preferred are hydrogels with a final storage moduli greater than 100, 200, 300, 400 Pa, or 1 kPa to form a stable hydrogel within the pericardial cavity for sustained release of encapsulated active agents. Large gauge catheters are able to access the pericardial cavity through a subxiphoid access, and therefore it is anticipated that highly crosslinked and stable hydrogels with storage moduli greater than lkPa and even 10 kPa can be delivered with clinically feasible injection forces of less than 100N, 50N or more preferably less than 25N.

Some preferred hydrogels have a storage modulus (G′) greater than the hydrogel's loss modulus (G″). This represents a more elastic hydrogel that is better able to withstand shear, compression, and even tensile forces in the dynamic environment within the pericardial cavity. In some embodiments the storage modulus is at least 5, 8, 10, 100, 200, 300, 400 Pa, or 1 kPa.

Hydrogels may enter the pericardial cavity with enough strength to create a clear boundary but continue to crosslink after injection to increase gel strength and stability, allowing localization of an encapsulated therapeutic agent. Further crosslinking or secondary crosslinking can be used to secure the gels in place through physical interdigitation with the epicardium. These in situ forming gels can be slowly crosslinking polymers that provide a sufficient window for injection after reaching a sufficient modulus for localization or can be quickly crosslinking polymers that are mixed over very short lengths before reaching the pericardial cavity.

In addition to physical interdigitation, the formation of covalent or non-covalent bonds between the hydrogel polymers and the epicardium or pericardium can be introduced to secure the gel at the injection site. These bonds include electrostatic interactions, van der Walls interactions, hydrophobic interactions, hydrogen bonding, and covalent bond formation including but not limited to aldehydes, carbodiimide, N-hydroxysuccinimide, maleimide, mussel derived adhesive chemistries such as catechols/gallols, and isocyanates, and enzyme mediated bond formation such as transglutaminase.

Shear-thinning hydrogels are crosslinked polymer networks that exhibit a reduction in mechanical integrity under the application of force. These materials include non-covalently crosslinked hydrogels as well as hydrogels with dynamic covalent crosslinks which have shown the ability to be injected through needles with clinically feasible injection forces for local delivery to diseased tissue, including but not limited to electrostatic and hydrophobic peptide and protein assemblies, guest-host chemistries such as cyclodextrin-adamantane, reversible covalent bonds such as schiff bases, hydrazones and oximes. Importantly, these materials do not clog the injection catheter, as they can be injected as a fully crosslinked network allowing for a larger time window to inject in a clinically setting. These materials exhibit injection forces of less than 100N, 50N and more preferably 25N through a needle. In addition, they may exhibit an injection force that varies by less than 5N, and more preferably less than 4N, 3N, 2N or 1N over the length of the syringe when the gel occupies the entire volume of the catheter. As little as 0.05 mL of hydrogel can be injected in this manner, or as much as 10 mL in an adult heart to cover a desired region of heart tissue. The preferred embodiment is between 1 mL and 5 mL of injected hydrogel.

Hydrogels are water-swollen polymer networks that exhibit many tissue-like properties and have been widely explored for tissue engineering and drug delivery applications. Hydrogels can form through numerous polymer crosslinking strategies including non-covalent interactions such as electrostatic, hydrogen, hydrophobic and van der Walls forces as well as covalent bond formation. Hydrogels can also be the result of physical entanglements of polymer chains. Crosslinking strategies that allow for hydrogel formation in situ, within the body, including thermal transitions and chemical bond formation with controllable kinetics are advantageous as they could potentially translate to catheter delivery for minimally invasive, percutaneous therapies. Further, shear thinning hydrogels and hydrogels with dynamic bonds allow injection of the crosslinked polymer network. As injection force is applied, the crosslinking bonds are broken, allowing the materials to be passed through a syringe or catheter. Crosslinking bonds may reform after injection as is the case with self-healing materials. The gel point is often described as the point where the storage modulus (G′) is greater than the loss modulus (G″) as measured by rheology. Materials where G′ is greater than G″ upon entering the pericardial cavity is the preferred embodiment.

These injectable hydrogels can serve as depots of encapsulated therapeutic agents for targeted delivery to organs such as the heart. Exogenous delivery of therapeutic agents including small molecules, peptides, cytokines, proteins, polysaccharides, synthetic polymers, particles, DNA plasmids, mRNA, cells, cellular exosomes and other cellular components can be used to treat the underlying mechanisms of organ disease such as heart disease; however, the high rates of diffusion, short in vivo half-lives, and potency make targeted delivery to specific organs such as the heart tissue challenging. To this end, injectable hydrogels provide a useful platform to localize and sustain the release of therapeutic agents to specific organs such as the heart. Molecules are encapsulated in the hydrogel matrix and released locally over time to sustain target levels of the molecule in the local vicinity of the hydrogel while preventing detrimental systemic effects. Molecule release can be controlled with polymer concentration, polymer-molecule interactions, polymer hydrophobicity, and hydrogel degradation. Hydrogel degradation can be controlled through crosslinking bond hydrolysis or polymer hydrolysis, but also in response to a stimulus such as light, pH, temperature or the presence of an enzyme. Further, nanoparticles and microparticles containing therapeutic agents can be encapsulated within hydrogels to provide additional control of therapeutic agent release.

We explored the potential to inject shear-thinning hydrogels into the pericardial space to localize encapsulated molecules to specific regions of the myocardium, such as a myocardial infarct. Briefly, aldehyde modified dextran was mixed with hydrazide modified gelatin and loaded into a syringe. Within minutes, the aldehyde and hydrazide functional groups began to react to form a crosslinked hydrogel within the syringe (FIG. 1). When the plunger on the syringe was pressed, the increased shear force breaks the aldehyde-hydrazide bonds, allowing passage through the needle. After allowing gels to crosslink for at least one hour, the needle was inserted into the pericardial cavity by positioning the needle at a very acute angle to the surface of the heart to avoid puncturing the myocardium. Hydrogel loaded with blue dye was injected into the cavity by applying a force to the syringe plunger. The hydrogel spread from the injection site as more material was dispensed but maintained a well defined boundary within the cavity. As a control, physiological buffer with blue dye was injected into the pericardial cavity, and the liquid quickly dispersed throughout the cavity in an uncontrolled manner, illustrating the importance of the hydrogel for localization.

Any suitable hydrogel may be utilized. In some embodiments, the hydrogel comprises at least one of hydrazide modified gelatin, hyaluronic acid, dextran, polyvinylpyrrolidone; methlycellulose and methlycellulose derivatives; polysaccharides; chitosan or polyethylene glycol and aldehyde modified hyaluronic acid, gelatin, dextran, polyvinylpyrrolidone; methlycellulose and methlycellulose derivatives; polysaccharides; chitosan or polyethylene glycol.

In some aspects, the methods are used to treat myocardial infarction, heart failure, atrial fibrillation, coronary artery disease, aetherosclerosis, angina, aneurysms, hypertension, rheumatic heart disease, cardiac arrest, ischemia, congestive heart failure, arrhythmia, congenital heart diseases, cardiomegaly, heart valve diseases, cardiomyopathy, dilated cardiomyopathy, hypertrophic cardiomyopathy, restrictive cardiomyopathy, pericarditis, pericardial effusion, marfan syndrome, heart murmers, or post surgical tissue repair.

In yet another aspect, the hydrogel composition prevents the formation of post-operative adhesions.

Certain embodiments utilize a hydrogel composition comprising a radiopaque material to guide the introduction of the hydrogel. Suitable radiopaque materials include iohexol or zirconia.

The pericardial cavity is the space between the epicardium—the outer surface of the heart muscle, and the pericardium—a fibrous casing that surrounds the heart. This cavity is filled with fluid, allowing the pericardium to move separately from the beating heart muscle. Pericardial injections and aspirations have been demonstrated in preclinical animal models as well as clinically. For example, fluid is often aspirated from the pericardial cavity in patients with pericarditis, an inflammation of the pericardium accompanied by increased fluid accumulation in the pericardial cavity. Pericardial fluid may be aspirated from the pericardial cavity prior to injecting hydrogels to create a tight boundary for hydrogel localization.

Various types of pharmaceuticals and compounds can be utilized with the instant hydrogels. For example, the inventive compositions may be used to treat atrial fibrillation. Suitable pharmaceuticals and compounds include histone deacetylase (HDAC) inhibitors such as hydroxamic acids (or hydroxamates), such as trichostatin A; cyclic tetrapeptides (such as trapoxin B), and the depsipeptides; benzamides; electrophilic ketones; aliphatic acid compounds such as phenylbutyrate and valproic acid; and any other zinc binding moiety.

The inventive compositions may be used to treat myocardial infarction. Suitable pharmaceuticals and compounds include matrix metalloproteinase (MMP) inhibitors such as recombinant tissue inhibitors of MMPs (TIMPs), including TIMP-1, TIMP-2, TIMP-3 and TIMP-4; hydroxymates including illomastat; tetracyclines including doxycycline, modified doxycyclines, minocycline; peptide based inhibitors; and other zinc binding moieties. Other myocardial infarction treatment agents include sulfated polysaccharides such as naturally produced molecules such as heparin, chondroitin sulfate, and dermatan sulfate; and semi-synthetically produced molecules such as pentosan polysulfate, sulfated hyaluronic acid, and dextran sulfate. Other treatment agents include (i) miRNA (to, for example, stimulate cellular processes such as angiogenesis, proliferation, differentiation, and production of extracellular matrix molecules such as collagen and glycosaminoglycans); (ii) siRNA (to, for example, stop production of inflammatory cytokines and proteases; (iii) plasmid DNA (to, for example, stimulate cellular processes such as angiogenesis, proliferation, differentiation, and production of extracellular matrix molecules such as collagen and glycosaminoglycans ; (iv) growth factors such as vascular endothelial growth factor (VEGF), insulin-like growth factor-1 (IGF-1) and hepatocyte growth factor (HGF), platelet-derived growth factor (PDGF), fibroblast growth factor (FGF), pleiotrophin, neuregulin; (v) heat shock proteins; (vi) cytokines (cell recruitment factors such as stromal derived cell factor 1 alpha (SDF-1α) or interferon gammas); (vii) cells such as adult stem cells such as mesenchymal stem cells (MSCs), hematopoietic stem cells (HSCs); induced pluripotent stem cells (iPS) from any tissue source; fibroblasts; cardiomyocytes; and endothelial cells; and (viii) cellular vesicles/exosomes.

Another treatment option is prevention of post-operative adhesions. Suitable pharmaceuticals and compounds include polyvinylpyrrolidone; polyethylene glycol; methlycellulose and methlycellulose derivatives such as carboxymethylcellulose; polysaccharides such as dextran, hyaluronic acid; chitosan and the hydrogel itself including hydrogel degradation products.

Further treatments include pericarditis. Suitable pharmaceuticals and compounds include (i) steroids such prednisone and (ii) anti-inflammatory compounds such as Interleukin 1 (IL-1) inhibitors such as IL-1 receptor antagonist and tetracyclines.

### Definitions

“Storage modulus” or (G′) is a measure of stored energy in a hydrogel subjected to force and represents the elastic portion of the complex modulus of viscoelastic materials. A higher G′ is indicative of a more solid-like hydrogel. G′ can be determined by oscillatory rheology with a 1 degree cone and plate at 0.5% strain, 1 Hz and at room temperature

“Loss modulus” or (G″) is a measure of energy dissipation under force and represents the viscous portion of the complex modulus of viscoelastic materials. G″ can be determined by oscillatory rheology with a 1 degree cone and plate at 0.5% strain, 1 Hz and at room temperature.

As used herein, the term “preformed” means that the hydrogel components are mixed prior to injection into the patient resulting in some level of polymer entanglement or crosslinking. In some embodiments, the hydrogel can be formed prior to it being placed within the syringe or catheter or, alternately, the hydrogel is formed within the syringe or catheter.

As used herein, the phrase “small molecule pharmaceutical” is a low molecular weight (<900 daltons) organic compound that may regulate a biological process.

The term “shear-thinning” means a reduction in viscosity of hydrogels under increasing shear stress.

It is also to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs.

As used in the specification and the appended claims, the singular forms “a,” “an” and “the” include plural referents unless the context clearly dictates otherwise. Thus, for example, reference to “a polyamide polymer” includes mixtures of two or more polyamide polymer

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optionally substituted alkyl” means that the alkyl group can or cannot be substituted and that the description includes both substituted and unsubstituted alkyl groups.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

The following examples are intended to be illustrative and not limiting.

### EXAMPLE 1

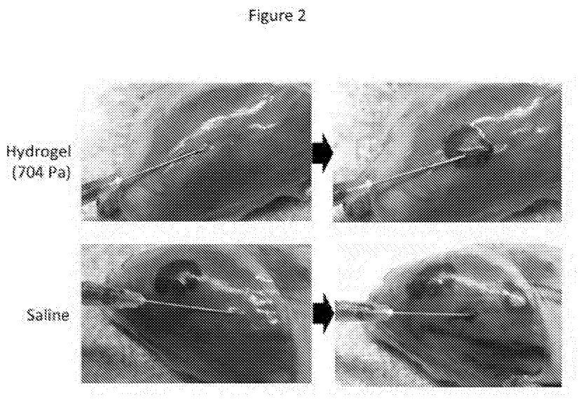

Aldehyde modified dextran was mixed with hydrazide modified gelatin and loaded into a syringe. Blue food coloring was added at 2% (v/v) for visualization purposes. After allowing gels to crosslink overnight, the 23G needle was inserted into the pericardial cavity of an explanted pig heart by positioning the needle at a very acute angle to the surface of the heart to avoid puncturing the myocardium (FIG. 2). Hydrogel was injected into the cavity by applying a force to the syringe plunger. The hydrogel spread from the injection site as more material was dispensed but maintained a well defined boundary within the cavity (FIG. 2). As a control, physiological buffer with 2% (v/v) blue food coloring was injected into the pericardial cavity, and the liquid quickly dispersed throughout the cavity in an uncontrolled manner. Hydrogel mechanics were quantified by injecting 50 mg of gel through a 23G needle and measured with a 1° cone (TA instruments, Part No. 511204.901) and plate oscillatory rheometry (TA instruments, AR2000ex) with 26 μm gap, 0.5% strain and 1 Hz. Storage modulus was 704 Pa.

### EXAMPLE 2

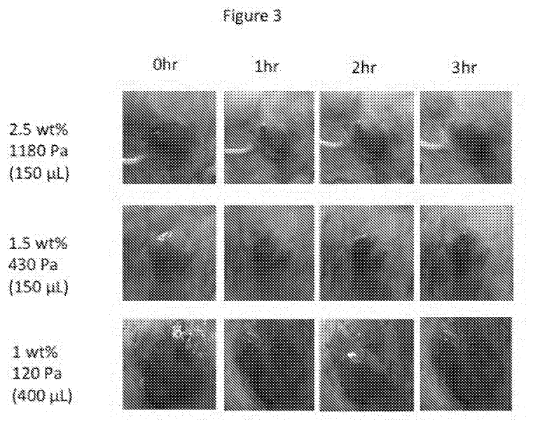

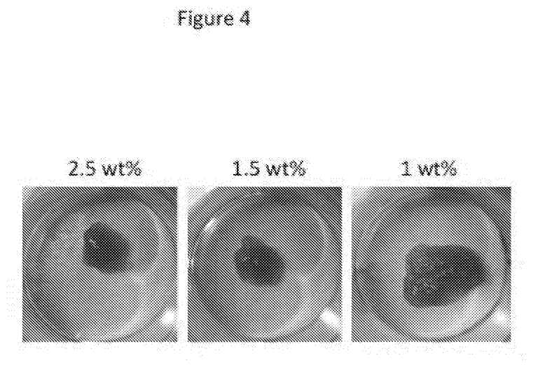

A beating heart model was constructed with explanted pig hearts to mimic in vivo myocardial wall motion. A compressed air source was used to inflate an elastic membrane attached to the end of plastic tubing. An electrically activated solenoid valve was used to vent the pressurized system and deflate the membrane once per second. The membrane and tubing was fed into the left ventricle of the explanted pig heart and the air source and valve were turned on to make the heart “beat”. The wall motion closely mimicked that of a beating heart as confirmed by a cardiologist. Aldehyde modified hyaluronic acid and hydrazide modified hyaluronic acid were mixed and aspirated into syringes at 2.5, 1.5 or 1 wt % and incubated overnight at room temperature. Hydrogel mechanics were quantified by injecting 50 mg of gel through a 23G needle and measured with a 1° cone (TA instruments, Part No. 511204.901) and plate oscillatory rheometry (TA instruments, AR2000ex) with 26 μm gap, 0.5% strain and 1 Hz. Storage moduli were 1180, 430, and 120 Pa for 2.5, 1.5 and 1 wt % gels respectively. Gels were incubated in the beating hearts for 3 hrs, after which they were explanted and examined. Saline was poured over the hearts every 15 min and the hearts were covered to maintain physiological moisture content. All gels remained intact and at the injection site over the course of the three hour beating model (FIG. 3). Gels could be explanted from the pericardial cavity as a singular gel that could be handled (FIG. 4).

### EXAMPLE 3

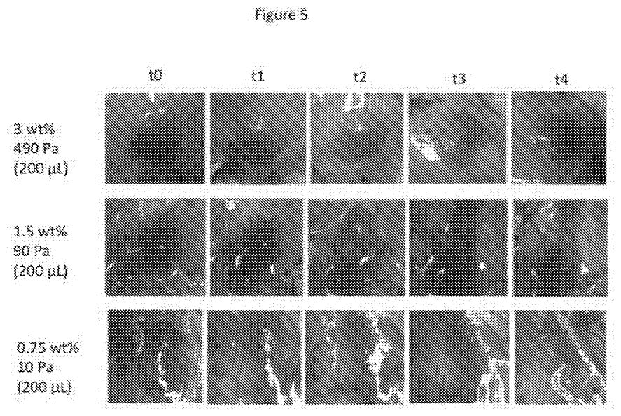

Aldehyde modified hyaluronic acid was mixed with hydrazide modified gelatin and loaded into syringes at a final polymer concentration of 0.75, 1.5 and 3 wt %. Gels were incubated overnight at room temperature. Hydrogel mechanics were quantified by injecting 50 mg of gel through a 18G needle and measured with a 1° cone (TA instruments, Part No. 511204.901) and plate oscillatory rheometry (TA instruments, AR2000ex) with 26 μm gap, 0.5% strain and 1 Hz. Storage moduli were 490, 90, and 10 Pa for 3, 1.5 and 0.75 wt % gels respectively. To mimic movement of the heart in the chest cavity and more specifically between the epicardium and pericardium, hydrogels were injected through 18G needles into the pericardial cavity of explanted pig hearts and exposed to pericardial shear motion by rubbing two fingers in a circular motion around the gel, moving the pericardium approximately 2 cm around the gel 10 times and imaged. This was repeated 4 times. All hydrogels formed a pocket within the pericardial cavity (FIG. 5). The 0.75 wt % gel moved and appeared to break apart during the pericardial shear motion. The 1.5 wt % gel moved but remained intact, while the 3 wt % gel remained as it was injected (FIG. 5).

### EXAMPLE 4

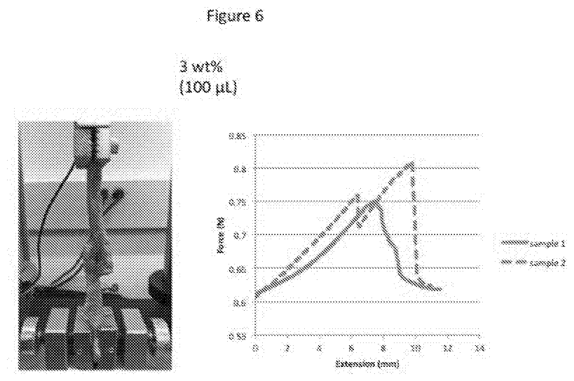

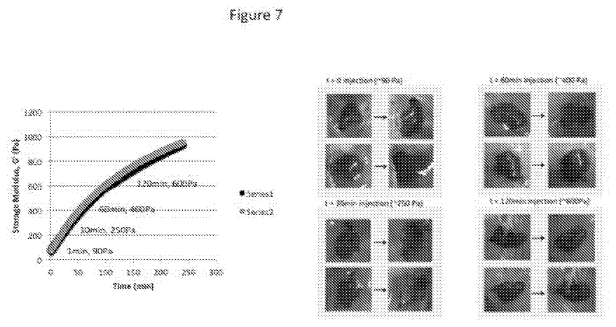

To measure adhesion of gels to the epicardium, 1 cm×5 cm strips of heart tissue with an epicardial surface were cut from explanted pig hearts. Aldehyde modified hyaluronic acid was mixed with hydrazide modified gelatin at a final polymer concentration of 3 wt % and 100 μL of the crosslinking polymer solution was pipetted between two strips of heart tissue, with an overlapping area of 1 cm×2 cm and epicardial surfaces facing each other and the gel. Tissues with gel were incubated for 3 hrs at room temperature and were covered in a container with saline to prevent the gels from dehydrating. Adhesion strength was measured by pulling the free ends of the tissues with an Instron at 10 mm/min. Force plots show adhesion of the gel with the epicardium (FIG. 6). Control tissues with 100 μL saline instead of gel showed little adhesion and were unable to be loaded into the Instron clamps without coming apart.

### EXAMPLE 5

Aldehyde modified hyaluronic acid was mixed with hydrazide modified gelatin at a final polymer weight percent of 3 percent. Gelation kinetics were measured with oscillatory cone and plate rheology (50 uL sample, 1° cone TA instruments, Part No. 511204.901, 26 μm gap, TA instruments, AR2000ex, 0.5% strain and 1 Hz). This gel formulation was also prepared in a syringe (18 gauge needle) and 200 μL injections into the pericardial cavity of explanted pig hearts approximately 1, 30, 60 and 120 minutes after mixing the aldehyde and hydrazide polymers. To mimic movement of the heart in the chest cavity and more specifically between the epicardium and pericardium, the gels were exposed to pericardial shear motion by rubbing two fingers in a circular motion around the gel, moving the pericardium approximately 2 cm around the gel 10 times and imaged. All gels remained intact during this motion.

### EXAMPLE 6

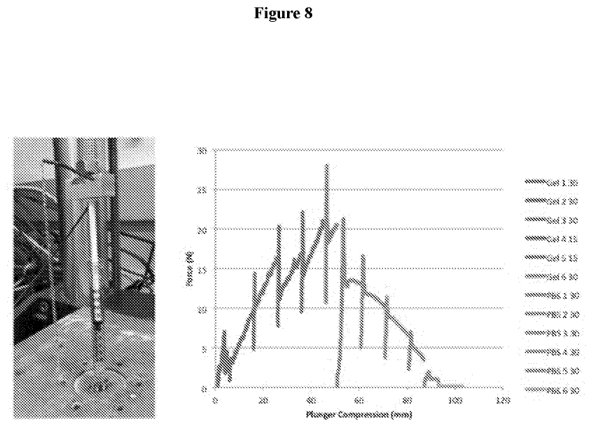

The hydrogel formulation from example 5 was prepared by mixing aldehyde modified hyaluronic acid and hydrazide modified gelatin at a final polymer weight percent of 3 percent. The polymers were incubated at room temperature in a syringe to crosslink for lhr, then loaded attached to a 5 Fr hydrophilic angle taper catheter (65 cm, GLIDECATH, Terumo). The syringe and catheter were loaded onto an Instron, and injection force was measured with compressive ramps of 15 mm/min and 30 mm/min until the syringe was empty (FIG. 8). Once the syringe was empty, a syringe with 1 mL of PBS was connected to the catheter and the force to flush out the gel with PBS was measured. The catheter has a void volume of approximately 0.7 mL.

### EXAMPLE 7

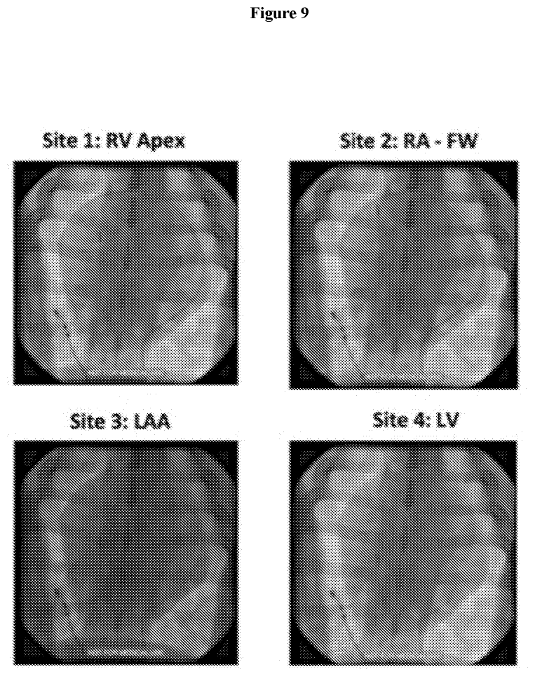

A male pig (75 kg) was sedated (ketamine, 22 mg/kg IM), intubated, and then maintained on 1.5-3% isoflurane (1.5 L/min). Percutaneous femoral arterial and venous access was obtained under ultrasound guidance and continuous hemodynamic monitoring with arterial blood pressure was performed and captured on a GE recording system. An intracardiac echo catheter was advanced and positioned in the right atrium and right ventricle for imaging and monitoring for complications. Atraumatic, percutaneous pericardial access was obtained under fluoroscopic access using an 18G Tuohy spinal needle through a subxiphoid incision without complication and a 5 Fr sheath was inserted into the pericardium. A 3 wt % hydrogel composed of aldehyde modified hyaluronic acid and hydrazide modified gelatin was prepared by mixing precursor polymers in a syringe (same formulation as examples 5 and 6), and the syringe was incubated at room temperature for 30 min before being injected into the 5 Fr hydrophilic angle taper catheter (65 cm, GLIDECATH, Terumo). After aspirating approximately 5 mL of pericardial fluid, the hydrogel loaded catheter was inserted into the pericardium and 0.3 ml of the hydrogel was injected at the right ventricle apex (Site 1) and lateral/FW right atrium (Site 2) (FIG. 9). There was no hemodynamic compromise observed. The catheter was navigated to the left atrium appendage region (Site 3) and lateral left ventricle (Site 4) for 2 additional 0.3 mL percutaneous injections (FIG. 9). There was no hemodynamic compromise observed. The catheter was removed and the hydrogel injected into the pericardium was allowed to instill for 60 minutes. Following this waiting period, median sternotomy was performed with the pericardium in-tact. The right sided injections were observed at the targeted locations.

### EXAMPLE 8

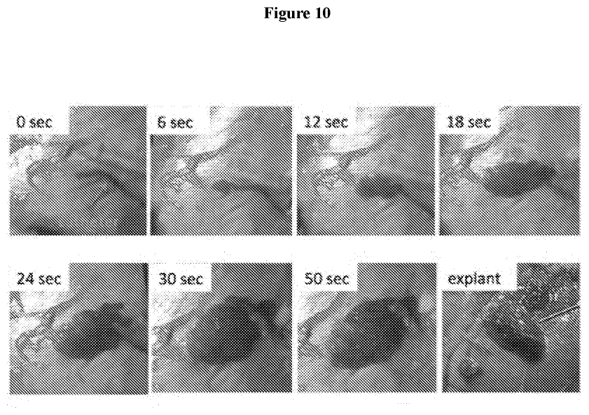

After performing the sternotomy and exposing the heart, the 5 Fr catheter was reinserted into the pericardial cavity and 1 mL of hydrogel was injected to visualize the injection. Mixed hydrogel was incubated for 2 hrs at room temperature prior to injection. The storage modulus of this material is approximately 2 kPa (50 mg gel injected through 18G needle, 1° cone TA instruments, Part No. 511204.901, 26 μm gap, TA instruments, AR2000ex, 0.5% strain and 1 Hz). The hydrogel gradually spread to form a single patch around the injection site (FIG. 10). After injection (˜24 seconds), the catheter was removed and hydrogel was observed for 5 min. The gel remained a solid patch at the injection site during myocardial wall motion. After euthanizing the pig, the heart was explanted and injected hydrogels were identified.

### EXAMPLE 9

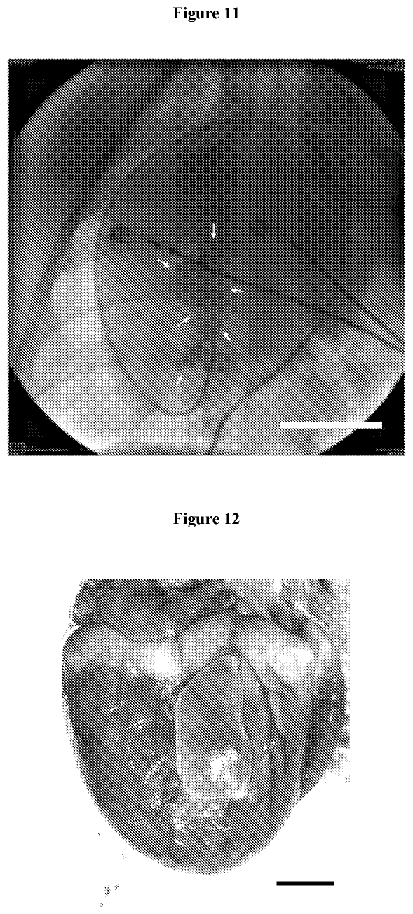

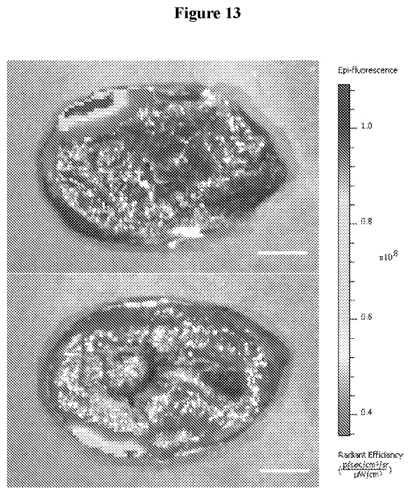

The percutaneous subxiphoid catheter procedure was performed in an adult pig and continuous hemodynamic monitoring was performed with arterial blood pressure and an intracardiac echo catheter was advanced and positioned in the right atrium and right ventricle for imaging and monitoring for complications. After gaining access to the pericardial cavity, 5 to 7 mL of pericardial fluid was removed. The 5 Fr catheter was then loaded with a 3 wt % hydrogel with encapsulated IR800 dye (10 mg/mL) and iohexol (100 mg/mL), and the catheter was guided to a target location on the heart using fluoroscopic guidance and 1.6 mL of hydrogel was injected. The mixed hydrogel was incubated for 15 min at room temperature prior to injection (G′ approximately 150 Pa). The hydrogel with a well-defined boundary could be visualized with fluoroscopy (FIG. 11). No hemodynamic compromise or atrial-ventricular complications were observed throughout the injection procedure. After allowing lhr for the gel to further crosslink, the pig was recovered. After 3 days, the pig was euthanized and the heart was explanted. Upon removing the pericardium, an adherent hydrogel patch was observed (FIG. 12). The hydrogel was peeled off of the heart and the left ventricle was sectioned and imaged with an IVIS Spectrum in order to visualize locally delivered IR800 (FIG. 13).

1. A method of delivering a hydrogel to the heart, comprising:
introducing a hydrogel composition into a subject, said hydrogel comprising components mixed prior to introduction;
the introducing being performed such that the hydrogel composition resides between the epicardium and the pericardium of the subject.

2. (canceled)

3. The method of claim 1, wherein the hydrogel has a storage modulus (G′) greater than about 10 Pa.

4. (canceled)

5. (canceled)

6. The method of claim 1, wherein at least a portion of the hydrogel crosslinking of the hydrogel is performed prior to injecting the introducing.

7. The method of claim 6, wherein the hydrogel composition is shear-thinning.

8. The method of claim 7, wherein the hydrogel is delivered with an injection force of less than 50N.

9. (canceled)

10. The method of claim 1, further comprising contacting two or more components to form the hydrogel composition.

11. The method of claim 10, wherein the contacting is performed in a mixer such that one hydrogel component is fed to the mixer through a first lumen of a catheter or syringe and a second hydrogel component is fed to the mixer through a second lumen of a catheter or syringe.

12. The method of claim 11, wherein a medicament is fed to the mixer by either the first or second lumen of a catheter or syringe.

13. The method of claim 11, additionally using a third lumen of a catheter or syringe to feed a medicament to the mixer.

14. The method of claim 1, wherein the hydrogel comprises at least one of hydrazide modified gelatin, hyaluronic acid, dextran, polyvinylpyrrolidone; methlycellulose and methlycellulose derivatives; polysaccharides; alginate, chitosan or polyethylene glycol and aldehyde modified hyaluronic acid, gelatin, dextran, polyvinylpyrrolidone; methlycellulose and methlycellulose derivatives; polysaccharides; alginate; chitosan or polyethylene glycol.

15. The method of claim 1, wherein the hydrogel composition contains a medicament of one or more of small molecule pharmaceuticals, peptides, cytokines, proteins, polysaccharides, synthetic polymers, particles, DNA plasmids, mRNA, cells, and cellular exosomes.

16. The method of claim 15, wherein the medicament comprises one or more of matrix metalloproteinase (MMP) inhibitors; hydroxymates; tetracyclines, minocycline; peptide based inhibitors; ion chelators.

17. (canceled)

18. The method of claim 16, wherein the hydroxymate comprises illomastat.

19. The method of claim 16, wherein the tetracycline comprises one or more of doxycycline, modified doxycyclines, and minocycline.

20. The method of claim 15, wherein the medicament comprises one or more of (i) miRNA; (ii) siRNA; (iii) plasmid DNA; (iv) growth factors; (v) heat shock proteins; (vi) cytokines; (vii) cells; and (viii) cellular vesicles/exosomes.

21. The method of claim 15, wherein the medicament comprises one or more of semi-synthetic sulfated polysaccharides.

22. The method of claim 15, wherein the medicament comprises one or more of naturally sulfated polysaccharides.

23. The method of claim 15, wherein the medicament comprises one or more of (i) steroids and (ii) anti-inflammatory compounds.

24. The method of claim 15, wherein the medicament comprises one or more metal ion chelators.

25. The method of claim 15, wherein the medicament comprises one of more histone deacetylase (HDAC) inhibitors.

26. The method of claim 1, wherein the method is used to treat myocardial infarction, heart failure, atrial fibrillation, coronary artery disease, atherosclerosis, angina, aneurysms, hypertension, rheumatic heart disease, cardiac arrest, ischemia, congestive heart failure, arrhythmia, congenital heart diseases, cardiomegaly, heart valve diseases, cardiomyopathy, dilated cardiomyopathy, hypertrophic cardiomyopathy, restrictive cardiomyopathy, pericarditis, pericardial effusion, Marfan syndrome, heart murmurs, post surgical tissue repair.

27. The method of claim 15, wherein the method is used to treat myocardial infarction, heart failure, atrial fibrillation, coronary artery disease, atherosclerosis, angina, aneurysms, hypertension, rheumatic heart disease, cardiac arrest, ischemia, congestive heart failure, arrhythmia, congenital heart diseases, cardiomegaly, heart valve diseases, cardiomyopathy, dilated cardiomyopathy, hypertrophic cardiomyopathy, restrictive cardiomyopathy, pericarditis, pericardial effusion, Marfan syndrome, heart murmurs, post surgical tissue repair.

28. The method of claim 1, wherein the hydrogel composition prevents the formation of post-operative adhesions.

29. The method of claim 15, wherein the hydrogel composition prevents the formation of post-operative adhesions.

30. The method of claim 1, wherein the hydrogel composition contains a radiopaque material to guide the introduction of the hydrogel.

31. (canceled)

32. The method of claim 15, wherein the hydrogel composition contains a radiopaque material to guide the introduction of the hydrogel.

33. (canceled)

34. The method of claim 1, wherein the hydrogel comprises contains chemical modifications adhesive groups to enhance adhesion to the epicardium or pericardium.

35. The method of claim 34, wherein the adhesive groups comprise aldehyde, catechol, or gallol groups.
